(19) United States
(12) Patent Application Publication
(10) Pub. No.: US 2014/0024907 A1
(43) Pub. Date: Jan. 23, 2014

(54) METHOD AND SYSTEM TO INDICATE HYPERGLYCEMIA OR HYPOGLYCEMIA FOR PEOPLE WITH DIABETES

(71) Applicants: Frances Wilson HOWELL, Chester Springs, PA (US); Janice MacLEOD, Chester Springs, PA (US); David RODBARD, Potomac, MD (US)

(72) Inventors: Frances Wilson HOWELL, Chester Springs, PA (US); Janice MacLEOD, Chester Springs, PA (US); David RODBARD, Potomac, MD (US)

(73) Assignee: LifeScan, Inc., Milpitas, CA (US)

(21) Appl. No.: 13/553,655

(22) Filed: Jul. 19, 2012

(51) Int. Cl. A61B 5/1477; A61M 5/168

(52) U.S. Cl. 600/347; 604/66

(57) ABSTRACT

Various methods and systems to manage diabetes of a subject using data relating to patterns to provide insight into how a patient's daily activities impact glycemic control of the subject. These patterns help to identify very specific areas of glycemic excursions, enable patients and HCPs to more easily identify patterns of hypoglycemia and hyperglycemia in order to take steps to improve glycemic control of the person with diabetes.

### BACKGROUND

Glucose monitoring is a fact of everyday life for many people with diabetes. The accuracy of such monitoring can significantly affect the health and ultimately the quality of life for people with diabetes. A person with diabetes may measure blood glucose levels several times a day as a part of the diabetes self management process. Failure to maintain target glycemic control can result in serious diabetes-related complications, including cardiovascular disease, kidney disease, nerve damage and blindness. There are a number of electronic devices currently available which enable an individual to check the glucose level in a small sample of blood. One such glucose meter is the OneTouch® Verio® glucose meter, a product which is manufactured by LifeScan.

In addition to glucose monitoring, people with diabetes often have to administer drug therapy such as insulin. People with diabetes self-administer insulin to manage their blood glucose concentration. There are a number of mechanical devices currently available which enable an individual to dose a predetermined quantity of insulin such as a hypodermic syringe, an insulin pen and an insulin pump. One such insulin pump is the Animas® Ping, a product which is manufactured by Animas Corporation. Another is the Animas® Vibe, also manufactured by Animas Corporation.

People with diabetes should maintain tight control over their lifestyle, so that they are not adversely affected by certain lifestyle choices such as irregular food consumption or exercise. In addition, a health care professional (HCP) dealing with a person with diabetes may require detailed information on the individual's lifestyle to provide effective treatment or modification of treatment for managing diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their HCP at each visit.

The aforementioned methods of recording lifestyle information are inherently difficult, time consuming and possibly inaccurate. Paper logbooks are not necessarily always carried by an individual and may not be accurately completed when required. Such paper logbooks are small and it is therefore difficult to enter the detailed information required of lifestyle events. Furthermore, an individual may often forget key facts about their lifestyle when questioned by a HCP who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system requires a laborious transcription of information, including lifestyle data, into this secondary data storage. Difficulty of data recordation encourages retrospective entry of pertinent information that results in inaccurate and incomplete records.

### SUMMARY OF THE DISCLOSURE

In one embodiment, a system for management of diabetes of a subject is provided. The system includes at least one glucose monitor, infusion pump and a controller for at least one of the monitor(s) and pump. The at least one glucose monitor is configured for measurements of the glucose levels of the subject. The insulin infusion pump is configured for communication with the at least blood glucose monitor and deliver insulin to the subject. The controller is in communication with at least the insulin infusion pump and/or the at least one glucose monitor and configured to receive or transmit data regarding glucose levels and dosing of insulin from the monitor and pump respectively, so that at least one of a hypoglycemic or hyperglycemic analysis of the subject is determined by: (a) evaluation of the glucose measurements for glucose measurements measured over distinct fasting periods for hypoglycemia or hyperglycemia during fasting, and in the event a first proportion of such blood glucose measurements measured over distinct fasting periods is less than a hypoglycemic threshold, provide an indication of hypoglycemia over a duration spanning the distinct fasting periods, or alternatively, in the event a second proportion of such blood glucose measurements measured over distinct fasting periods is greater than a hyperglycemic threshold, annunciating an indication of hyperglycemia over a duration spanning the distinct fasting periods; and (b) ascertainment from the glucose measurements for glucose measurements measured after external events have occurred to the user for hypoglycemia or hyperglycemia after such external events and provide an indication of hypoglycemia or hyperglycemia whenever a portion of the blood glucose measurements measured after the external disturbance is at or below a hypoglycemic threshold or whenever the blood glucose measurements measured after the external disturbance is at or above a hyperglycemic threshold.

In another embodiment, a method for managing diabetes of a subject with at least a blood glucose monitor is provided by applicants. The method can be achieved by: conducting, with the blood glucose monitor, a plurality of blood glucose measurements of the subject; storing the plurality of blood glucose measurements in a memory; and evaluating the plurality of blood glucose measurements measured over distinct fasting periods for hypoglycemia or hyperglycemia during fasting, and in the event a first proportion of such blood glucose measurements measured over distinct fasting periods is less than a hypoglycemic threshold, annunciating an indication of hypoglycemia over a duration spanning the distinct fasting periods, or alternatively, in the event a second proportion of such blood glucose measurements measured over distinct fasting periods is greater than a hyperglycemic threshold, annunciating an indication of hyperglycemia over a duration spanning the distinct fasting periods. From this method, the evaluation includes: determining a number N1 of blood glucose measurements from the blood glucose measured over distinct fasting periods in which each of the N1 number of blood glucose measurements is at or below a hypoglycemic threshold; calculating hypoglycemic value V1 which is approximately the number N1 divided by the number of the blood glucose measurements over distinct fasting periods and multiplied by 100; and annunciating hypoglycemia of the user during duration covering the distinct fasting periods whenever N1 is equal to or greater than a first predetermined value and the value V1 is equal to or greater than a second predetermined value. The evaluating may include determining a number N2 of blood glucose measurements from the blood glucose measured over distinct fasting periods in which each of the N2 number of blood glucose measurements is at or above a hyperglycemic threshold; calculating hyperglycemic value V2 which is approximately the number N2 divided by the number of the blood glucose measured over distinct fasting periods and multiplied by 100; and annunciating hyperglycemia of the user during duration covering the distinct fasting periods whenever N2 is equal to or greater than a third predetermined value and the value V1 is equal to or greater than a fourth predetermined value.

In a further embodiment, a method for managing diabetes of a subject with at least a blood glucose monitor is provided. The method can be achieved by: conducting, with at least the blood glucose monitor, a plurality of blood glucose measurements of the subject; storing the plurality of blood glucose measurements in a memory; ascertaining from the plurality of blood glucose measurements measured after external disturbances to the user for hypoglycemia or hyperglycemia after such external disturbances and annunciating an indication of hypoglycemia or hyperglycemia whenever a portion of the blood glucose measurements measured after the external disturbance is at or below a hypoglycemic threshold or whenever the blood glucose measurements measured after the external disturbance is at or above a hyperglycemic threshold.

In yet a further embodiment, a method of managing diabetes can be achieved by: ascertaining from the plurality of blood glucose measurements for blood glucose measurements measured within a predetermined time after delivery of insulin to the user for hypoglycemia or hyperglycemia after such insulin delivery; and annunciating an indication of hypoglycemia or hyperglycemia whenever a portion of the blood glucose measurements measured after the insulin delivery is at or below a hypoglycemic threshold or whenever the blood glucose measurements measured after the insulin delivery is at or above a hyperglycemic threshold. The ascertaining may include: determining from the blood glucose measurements taken within a predetermined time period after the external event in the form of insulin delivery, a first post-insulin BG number (I1) of blood glucose measurements that are at or below a hypoglycemic threshold; calculating a post-insulin hypoglycemic value (IN1) which is approximately the first post-insulin number (I1) of blood glucose measurements divided by a total number of blood glucose measurements measured after each insulin dosing and multiplied by 100; and annunciating hypoglycemia of the user after the external events in the form of insulin dosing whenever the hypoglycemic value (IN1) is greater than a predetermined value. Alternatively, the ascertaining may include: determining from the blood glucose measurements taken within a predetermined time period after insulin dosing, a second post-insulin number (I2) of blood glucose measurements that are at or above a hyperglycemic threshold; calculating a hyperglycemic value (IN2) which is approximately the second post-insulin number (I2) of blood glucose measurements divided by a total number of blood glucose measurements measured after the insulin dosing and multiplied by 100; and annunciating hyperglycemia of the user after the external events in the form of insulin dosing whenever the hyperglycemic value (IN2) is greater than another predetermined value.

In another embodiment, a method for managing diabetes via analysis of external disturbances or external events can be achieved by: conducting, with at least the blood glucose monitor, a plurality of blood glucose measurements of the subject; storing the plurality of blood glucose measurements in a memory; ascertaining from the plurality of blood glucose measurements for blood glucose measurements measured after external events have occurred to the user for hypoglycemia or hyperglycemia after such external events and annunciating an indication of hypoglycemia or hyperglycemia whenever a portion of the blood glucose measurements measured after the external disturbance is at or below a hypoglycemic threshold or whenever the blood glucose measurements measured after the external disturbance is at or above a hyperglycemic threshold. The ascertaining may include determining from the blood glucose measurements taken within a predetermined time period after each distinct external disturbance a first number (IEE1) of blood glucose measurements that are at or below a hypoglycemic threshold; calculating a hypoglycemic value (INE1) which is approximately the first number (IEE1) of blood glucose measurements divided by a total number of blood glucose measurements measured after each external disturbance and multiplied by 100; and annunciating hypoglycemia of the user after external events whenever the hypoglycemic value (INE1) is greater than a predetermined value. Alternatively, the ascertaining may include determining from the blood glucose measurements taken within a predetermined time period after each distinct external disturbance a second number (IEE2) of blood glucose measurements that are at or above a hyperglycemic threshold; calculating a hyperglycemic value (INE2) which is approximately the second number (IEE2) of blood glucose measurements divided by a total number of blood glucose measurements measured after each external disturbance and multiplied by 100; and annunciating hyperglycemia of the user after external events whenever the hyperglycemic value (INE2) is greater than another predetermined value.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE FIGURES

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 illustrates in schematic form the software engine to determine hypoglycemia or hyperglycemia of a subject based on input data from either or both of at least a glucose monitor and an insulin infusion pump.

FIG. 2 illustrates an exemplary glucose management system that can be used with the software engine of FIG. 1.

FIG. 3 illustrates, as an example, a logical process for hypoglycemia or hyperglycemia fasting patterns 300 or 400 of the software engine 200 in FIG. 1.

FIG. 4 illustrates, as an example, a logical process for hypoglycemia or hyperglycemia patterns for post-insulin glucose response patterns 500 or 600 of the software engine 200 in FIG. 1.

FIG. 5 illustrates, as an example, a logical process for hypoglycemia or hyperglycemia patterns 700 or 800 due to external disturbances or events of the software engine 200 in FIG. 1.

FIG. 6 illustrates, as an example, an annunciation of the pattern recognitions of FIGS. 3-5 as a display screen, which could also be printed out in paper media.

### MODES FOR CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment.

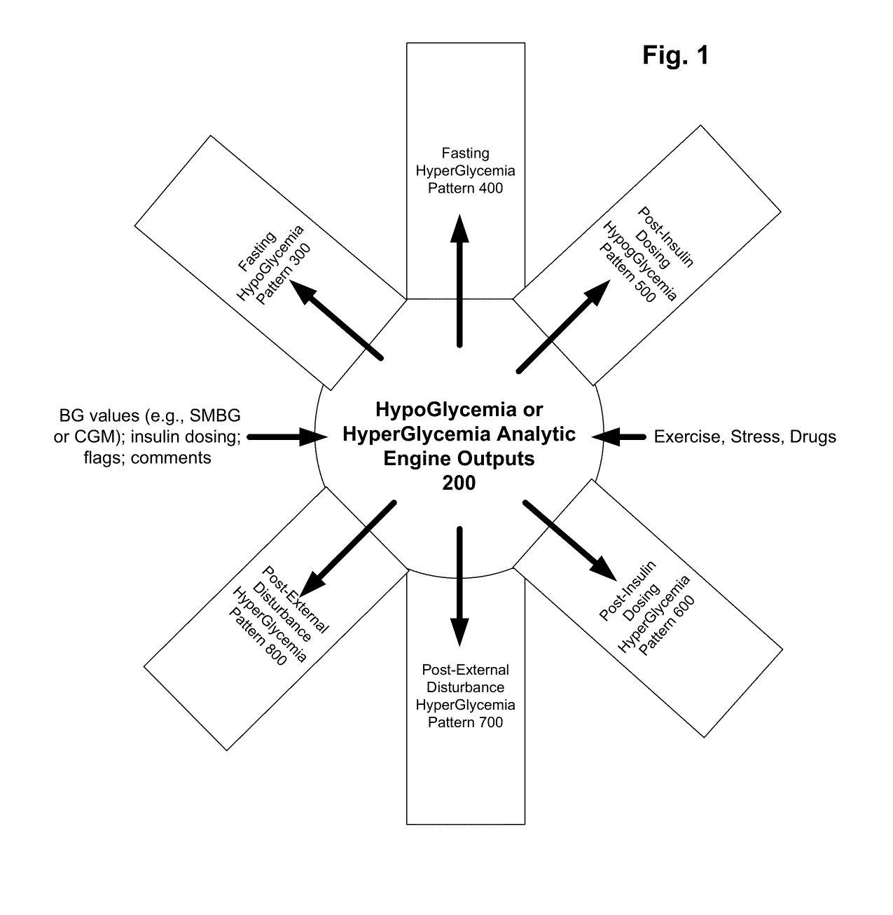

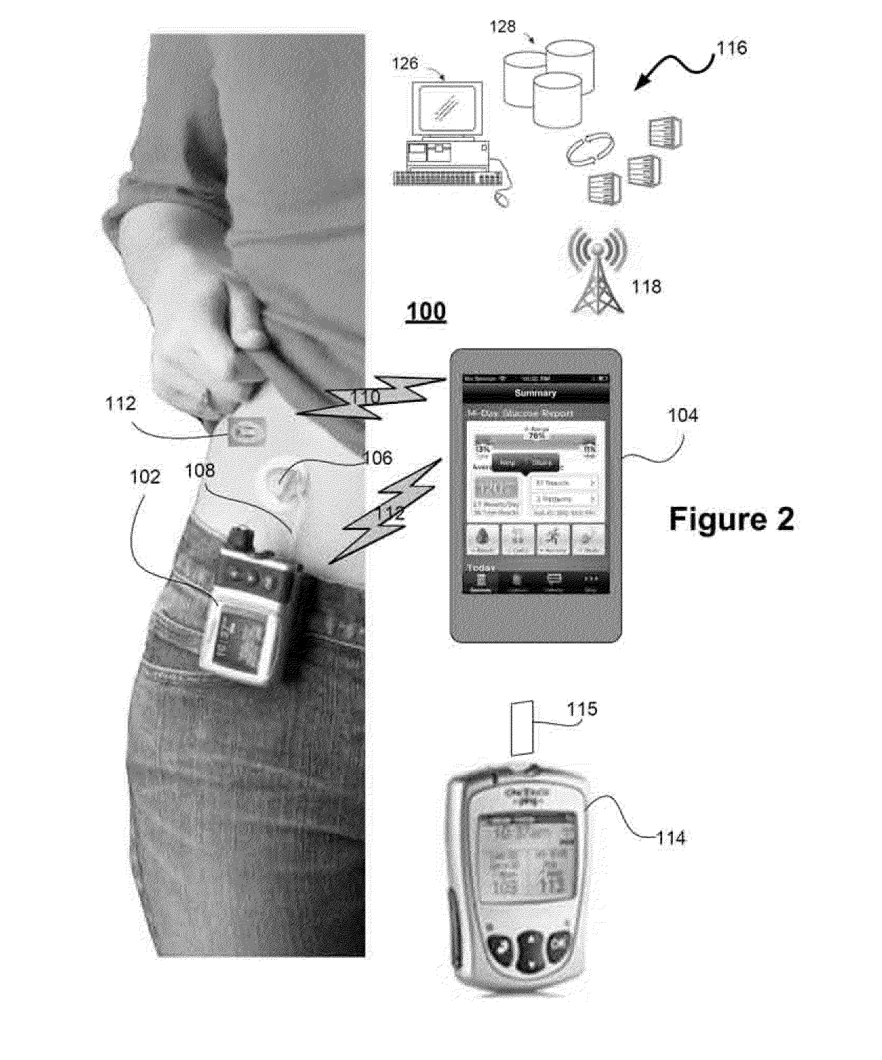

FIG. 1 illustrates a software engine 200 configured for use with microprocessor-enabled components of the FIG. 2. The software engine 200 receives a plurality of inputs to determine hypoglycemia (i.e., low blood glucose) and hyperglycemia (i.e., high blood glucose) of a subject. In particular, the inputs to the engine 200 may include blood glucose (“BG”) values derived from either a discontinuous glucose monitor (e.g., glucose test meter and strips) or a continuous glucose monitor. Other inputs may include insulin delivered to the subject's body, physical or mental stress applied on the subject, including environmental factors or drugs (hereafter “external events or external disturbances”). The engine 200 is configured to detect various patterns from the inputs such as, for example, hypoglycemic pattern 300 or hyperglycemic pattern 400 that may occur during a fasting state (i.e., a state in which the subject has not eaten any food for at least 6 hours), hypoglycemia pattern 500 after insulin dosing, hyperglycemia pattern 600 after insulin dosing, post external disturbance hypoglycemia pattern 700, or post external disturbance hyperglycemia pattern 800.

FIG. 2 illustrates a drug delivery system 100 according to an exemplary embodiment. Drug delivery system 100 includes a drug delivery device 102 and a remote controller 104. Drug delivery device 102 is connected to an infusion set 106 via flexible tubing 108. Drug delivery device 102 is configured to transmit and receive data to and from remote controller 104 by, for example, radio frequency communication 110. Drug delivery device 102 may also function as a stand-alone device with its own built in controller.

In one embodiment, drug delivery device 102 may include a drug infusion device and remote controller 104 may include a hand-held portable controller. In such an embodiment, data transmitted from drug delivery device 102 to remote controller 104 may include information such as, for example, drug delivery data, blood glucose information, basal insulin delivery, bolus insulin delivery, insulin to carbohydrates ratio or insulin sensitivity factor, to name a few. The controller 104 is configured to include a controller that has been programmed to receive continuous analyte readings from a CGM sensor 112. Data transmitted from remote controller 104 to drug delivery device 102 may include analyte test results and a food database to allow the drug delivery device 102 to calculate the amount of drug to be delivered by drug delivery device 102. Alternatively, the remote controller 104 may perform basal dosing or bolus calculation and send the results of such calculations to the drug delivery device. In an alternative embodiment, an episodic blood analyte meter 114 may be used alone or in conjunction with the CGM sensor 112 to provide data to either or both of the controller 102 and drug delivery device 102. Alternatively, the remote controller 104 may be combined with the meter 114 into either (a) an integrated monolithic device; or (b) two separable devices that are dockable with each other to form an integrated device. Each of the devices 102, 104, and 114 has a suitable micro-controller (not shown for brevity) programmed to carry out various functionalities.

Drug delivery device 102 may also be configured for bi-directional wireless communication with a remote health monitoring station 116 through, for example, a wireless communication network 118. Remote controller 104 and remote monitoring station 116 may be configured for bi-directional wired communication through, for example, a telephone land based communication network. Remote monitoring station 116 may be used, for example, to download upgraded software to drug delivery device 102 and to process information from drug delivery device 102. Examples of remote monitoring station 116 may include, but are not limited to, a personal or networked computer 126, server 128 to memory storage, a personal digital assistant, other mobile telephone, a hospital base monitoring station or a dedicated remote clinical monitoring station.

Drug delivery device 102 includes certain components including a central processing unit, memory elements for storing control programs and operation data, a radio frequency module 116 for sending and receiving communication signals (i.e., messages) to/from remote controller 104, a display for providing operational information to the user, a plurality of navigational buttons for the user to input information, a battery for providing power to the system, an alarm (e.g., visual, auditory or tactile) for providing feedback to the user, a vibrator for providing feedback to the user, and a drug delivery mechanism (e.g. a drug pump and drive mechanism) for forcing a drug from a drug reservoir (e.g., a drug cartridge) through a side port connected to an infusion set 106 and into the body of the user. Other suitable infusers can also be utilized such as, for example, a basal and bolus patch pump or even an infusing pen can also be utilized.

Analyte levels or concentrations can be determined by the use of the CGM sensor 112. The CGM sensor 112 utilizes amperometric electrochemical sensor technology to measure analyte levels with three electrodes operably connected to the sensor electronics and are covered by a sensing membrane and a biointerface membrane, which are attached by a clip.

The top ends of the electrodes are in contact with an electrolyte phase (not shown), which may include a free-flowing fluid phase disposed between the sensing membrane and the electrodes. The sensing membrane may include an enzyme, e.g., analyte oxidase, which covers the electrolyte phase. In this exemplary sensor, the counter electrode is provided to balance the current generated by the species being measured at the working electrode. In the case of an analyte oxidase based glucose sensor, the species being measured at the working electrode is H₂O₂. The current that is produced at the working electrode (and flows through the circuitry to the counter electrode) is proportional to the diffusional flux of H₂O₂. Accordingly, a raw signal may be produced that is representative of the concentration of blood glucose in the user's body, and therefore may be utilized to estimate a meaningful blood glucose value. Details of the sensor and associated components are shown and described in U.S. Pat. No. 7,276,029, which is incorporated by reference herein as if fully set forth herein this application. In one embodiment, a continuous analyte sensor from the Dexcom Seven System (manufactured by Dexcom Inc.) can also be utilized with the exemplary embodiments described herein.

In one embodiment of the invention, the following components can be utilized as a diabetes management system: microprocessor enabled device such as a home computer or a portable handheld computer (e.g., iPhone, iPad, or Android based devices) specifically programmed to receive data from multiple sources (e.g., exercise machine or other sensors) including at least an episodic glucose sensor with test strips such as the Verio blood glucose meter manufactured by LifeScan Inc. or DexCom® SEVEN PLUS® CGM by DexCom Corporation. The microprocessor-enabled device is specifically programmed so that such microprocessor-enabled device is converted into a purpose built diabetes management computer when placed in such mode of operation.

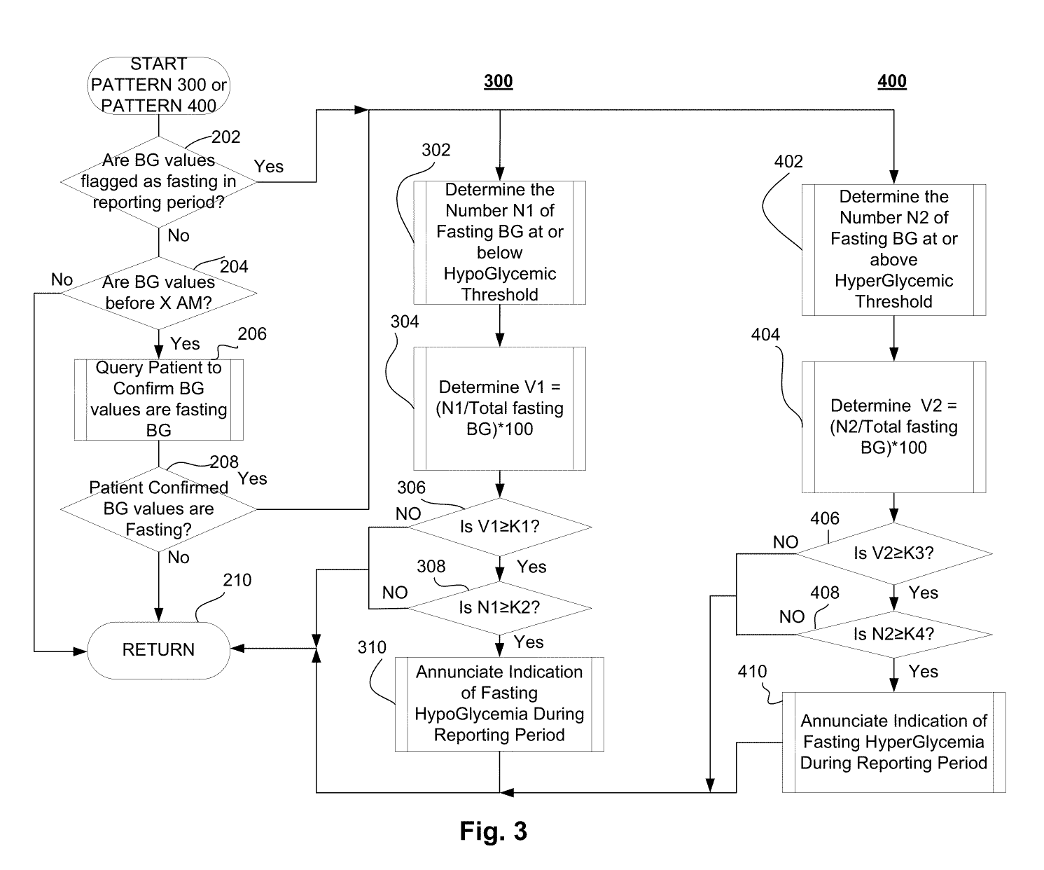

FIG. 3 illustrates an exemplary logic process for hypoglycemic (i.e., low blood glucose) pattern 300 or hyperglycemic (high blood glucose) pattern 400. At step 202, the logic determines whether blood glucose data from the episodic glucose monitor or continuous glucose monitor have been flagged as “fasting” when such glucose measurements were taken. As used herein, the term “fasting” in relation to blood glucose measurement means that the subject has not eaten any appreciable amount of food/caloric liquids for at least 6 hours prior to the measurement of the glucose concentration in the subject. If the query at 202 is false, meaning that the BG values have not been flagged, the logic will attempt to infer whether some or all of the BG values were taken while the subject was fasting. One technique may include making the assumption that any glucose measurement before a certain time in the morning is a glucose measurement made while fasting. This assumption can be further refined by the logic reviewing the pre-meal and post-meal flags to infer the time at the first meal in the morning is typically taken and setting such time as a threshold for inferring fasting glucose measurements made before such time threshold. To further validate the inference, the logic may include step 206 to require the subject to confirm whether some or all of the BG values obtained are actual fasting glucose measurements. Once the subject has confirmed at step 208 that certain of the stored BG values are fasting, the logic proceeds to determine fasting hypo in pattern 300 or fasting hyper in pattern 400. In situations where the subject may have tested multiple times within a short span (e.g., multiple episodic tests within 30 minutes) of the time period under scrutiny or when continuous glucose data is provided every few minutes, the logic will use an average of the episodic or CGM glucose values.

Another technique to infer fasting glucose without the use of flags by the subject is shown and described in US Patent Application Publication No. 2009-0240127 (Ser. No. 12/052,639), which is hereby incorporated by reference as if fully set forth herein this application.

Returning back to step 202 of FIG. 3, if the query at 202 is true then the logic moves to either or both of pattern determinations 300 or 400. For hypoglycemic pattern determination 300, the logic proceeds at step 302 to determine the number N1 indicative of blood glucose measured and flagged fasting by the user and which have a value at or below a hypoglycemic threshold. Step 304 calculates the proportion V1 of such number N1 of hypoglycemic measurements with respect to the total number of blood glucose measurements made while fasting. If both the proportion V1 is greater than a first predetermined constant K1 and the number of flagged hypoglycemic measurements N1 is at or greater than a second predetermined constant K2 then the logic annunciates to the subject, caretaker or health care provider that in a reporting period the subject has experienced hypoglycemia during fasting periods. In the preferred embodiments, the hypoglycemic threshold includes any value at or below a normal range such as, for example, 70 mg/dL. It is noted that for glucose measurements that have been tagged by the patient as taken before a meal (“pre-meal”), the range may be configured to be from about 70 mg/dL to about 130 mg/dL whereas measurements that have been tagged as measured after a meal (“post-meal”), the range may be configured to be from about 70 mg/dL to about 180 mg/dL. If the measurements have not been tagged by the patient then the range is configured to be from about 70 mg/dL to about 180 mg/dL.

For hyperglycemic pattern determination 400 in FIG. 3, the logic proceeds at step 402 to determine the number N2 indicative of blood glucose measured and flagged fasting by the user and which have a value at or below a hyperglycemic threshold. Step 404 calculates the proportion V2 of such number N2 of hypoglycemic measurements with respect to a total number of blood glucose measurements made while fasting. If both the proportion V2 is greater than a third predetermined constant K3 and the number of flagged hyperglycemic measurements N2 is at or greater than a fourth predetermined constant K4 then the logic annunciates to the subject, caretaker or health care provider that in a reporting period the subject has experienced hyperglycemia during fasting periods. In the preferred embodiments, the hyperglycemic threshold include any value at or above a normal range, such as, for example, any value from about ≧180 mg/dL, K1 is set to about 5 and K2 is set to about 2, K3 is set to about 50, and K4 is set to about 3.

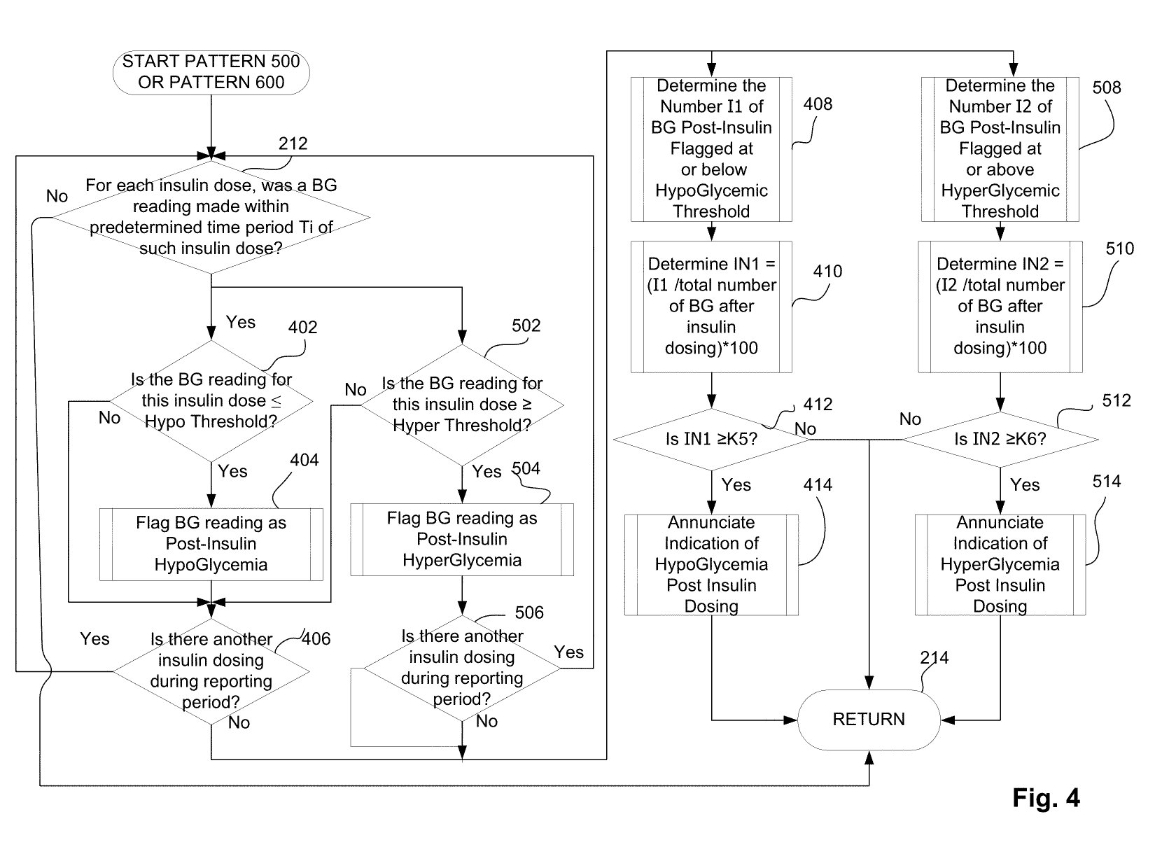

Assuming that the subject, caretaker or healthcare provider is interested in determining the glycemic responses of the subject to dosing of the subject with insulin during a given reporting period, then pattern 400 or pattern 500 may be used for such purposes. In FIG. 4, the logic looks to the memory of the controller, meter or pump to determine when insulin doses were given to the subject (as recorded or flagged by the subject or stored by the pump). For each dose of insulin, the logic determines at step 212 whether there was a BG measurement made within a predetermined time period Ti. If true, the logic may evaluate such BG value for its hypoglycemic or hyperglycemic impact. At step 402, the system determines the hypoglycemic impact by inquiring whether this specific BG is less than or equal to an insulin-related hypoglycemic threshold and if true, such BG is flagged as insulin related hypoglycemia at step 404. Thereafter, the logic flows to step 406 to determine whether another insulin dosing should be considered for hypoglycemic impact and so on until all insulin dosings have been considered. In situations where the subject may have tested multiple times within a short span (e.g., multiple episodic tests within 30 minutes) of the time period under scrutiny or when continuous glucose data is provided every few minutes, the logic will use an average of the episodic or CGM glucose values within the time period Ti for evaluation with respect to the hypo or hyper thresholds.

The system may also consider the hyperglycemic impact of insulin related dosings. In particular, at step 502, the system determines the hyperglycemic impact by inquiring whether this specific BG is equal to or greater than an insulin-related hyperglycemic threshold and if true, such BG is flagged as insulin related hyperglycemia at step 504. Thereafter, the logic flows to step 506 to determine whether another insulin dosing should be considered for hyperglycemic impact and so on until all insulin dosings have been considered.

Once the delivered doses of insulin (“insulin dosings”) have been linked to BG values reflective of hypoglycemia or hyperglycemia within the predetermined time period Ti, a determination can be made of whether such BG values over a reporting time period have risen to a level indicative of hypoglycemia or hyperglycemia in the user. For hypoglycemia (i.e., low blood glucose), the logic determines, at step 408, the number I1 of after insulin dosing flagged at or below the hypoglycemic threshold. Step 410 determines a proportion IN1 where IN1 is approximately the number I1 divided by the total number of BG measurements made after insulin dosings within time frame Ti of each dose and multiplied by 100. At step 412, if the proportion IN1 is about equal to or greater than a fifth constant K5 then the logic prompts the microprocessor to annunciate an indication of hypoglycemia after insulin dosings during a selected reporting period. If step 412 returns a false, the logic returns to the main routine at step 214.

For hyperglycemia (i.e., high blood glucose), the logic determines at step 508 the number I2 of after insulin dosing flagged at or above the hyperglycemic threshold. Step 510 determines a proportion IN2 where IN2 is approximately the number I2 divided by the total number of BG measurements made after insulin dosings within time frame Ti of each dose and multiplied by 100. At step 512, if the proportion IN2 is about equal to or greater than a sixth constant K6 then the logic prompts the microprocessor to annunciate an indication of hyperglycemia after insulin dosings during a selected reporting period. If step 512 returns a false logical state, the logic returns to the main routine at step 214. In the preferred embodiments, the predetermined time Ti is about 1.5 hours to about 4 hours, constant K5 is about 5 and the constant K6 is about 50.

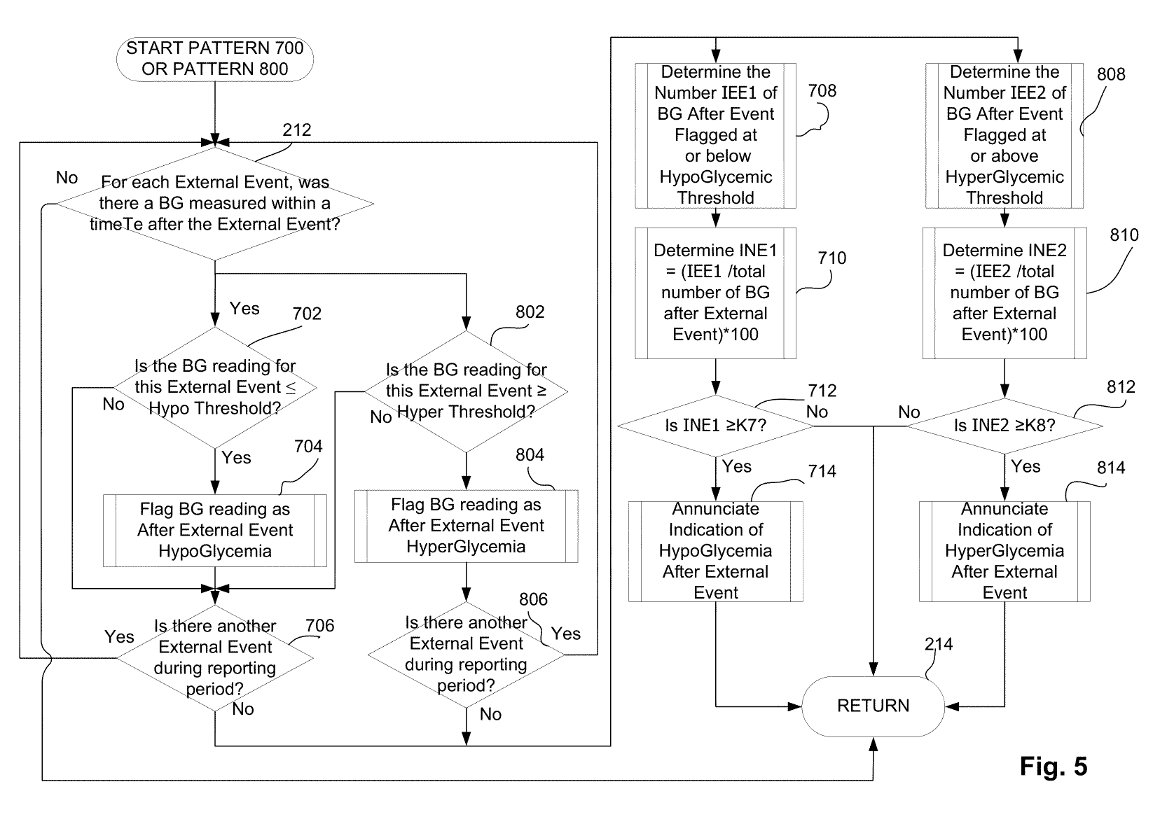

The system is also configured to consider other external events or external disturbances to the subject that could affect the glycemic state of the subject such as, for example, exercise, physical stress, mental stress, other drugs or illness. The determination of hypo pattern or hyper pattern can be ascertained with the logic of FIG. 5.

In FIG. 5, the logic considers each external disturbance or event that is reported to the system from physiological sensors or other data collection devices. The external event may include exercises reported by the subject or by exercise machines or sensors, or other data input from a health care provider or medical records. In order to be linked to the external event, the measured BG must have been made at a predetermined time period Te after the occurrence of the external event. This determination is made at step 212. Once a measured BG has been determined as occurring after an external disturbance the system, at step 502, determines the hypoglycemic or hyperglycemic impact in steps 702 and 802. In step 702 the logic queries its stored BG values to determine whether a specific BG is equal to or less than external-event-related hypoglycemic threshold, and if true, such BG if flagged as external disturbance related hypoglycemia. In step 802, the logic considers whether the particular BG is equal to or greater than an external event related hyperglycemic threshold and if true, such BG is flagged as insulin related hyperglycemia. Thereafter, the logic flows to respective pattern determination for hypo pattern in steps 708-714 or hyper pattern in steps 808-814.

Once the external event has been linked to BG values reflective of hypoglycemia or hyperglycemia in steps 702-706 or 802-806, determination can be made of whether such BG values have risen to a level indicative of hypoglycemia or hyperglycemia in the user over a given reporting period. For hypoglycemia (i.e., low blood glucose), the logic determines at step 708 the number IEE1 of the external events that have been flagged at or below the hypoglycemic threshold. Next, step 710 determines a proportion INE1 where INE1 is approximately the number IEE1 divided by the total number of BG measurements made after the external event within time frame Te of each event and multiplied by 100. At step 712, if the proportion INE1 is about equal to or greater than a seventh constant K7 then the logic prompts the microprocessor to annunciate an indication of hypoglycemia after external events during a selected reporting period. If step 712 returns a false logic state, the logic returns to the main routine at step 214.

For hyperglycemia (i.e., high blood glucose), the logic determines at step 808 the number IEE2 of the external events that have been flagged at or above the hyperglycemic threshold. Step 810 determines a proportion INE2 where INE2 is approximately the number IEE2 divided by the total number of BG measurements made after the external event within time frame Te of each dose and multiplied by 100. At step 812, if the proportion INE2 is about equal to or greater than eight constant K8 then the logic prompts the microprocessor to annunciate an indication of hyperglycemia after the external events during a selected reporting period. If step 812 returns a false, the logic returns to the main routine at step 214. In the preferred embodiments, the reporting period may be any suitable range from about 5 days to about 270 days, the time period Te may be about 4 hours or less, constant K7 is about 5 and the constant K8 is about 50. In situations where the subject may have tested multiple times within a short span (e.g., multiple episodic tests within 30 minutes) of the time period under scrutiny or when continuous glucose data is provided every few minutes, the logic will use an average of the episodic or CGM glucose values collected within the time period Te for each of the external disturbances in the evaluation with respect to the hypo or hyper thresholds.

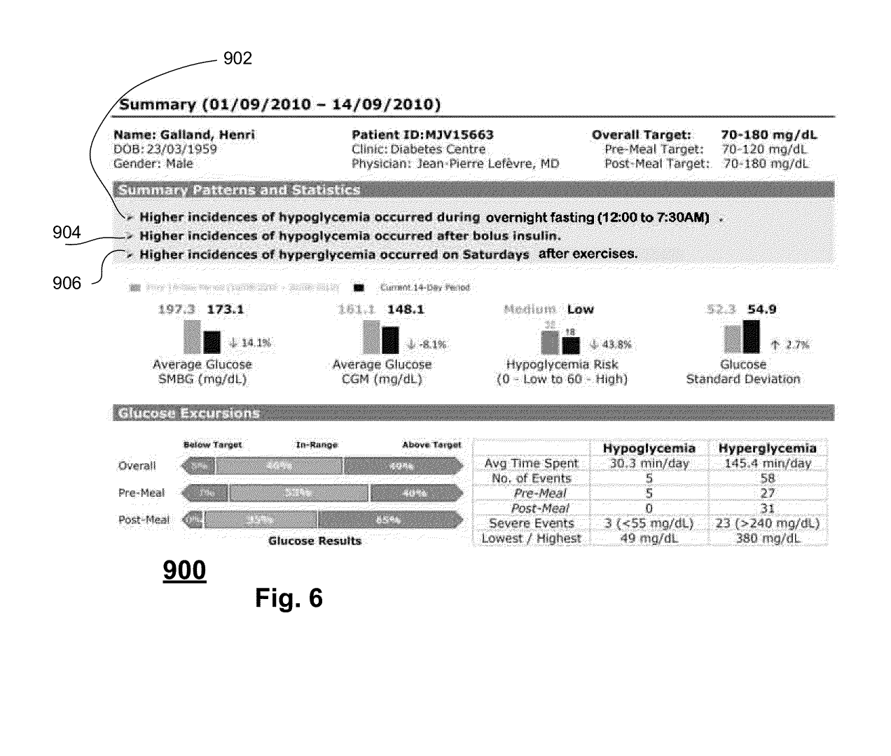

It is noted that recommendations, warnings and compliance updates may be annunciated to a user in a suitable medium, such as a visual medium in the form of a display screen, printed paper, or in the form of an audio message to the user or subject. In one embodiment, as shown in FIG. 6, a display screen can be utilized to annunciate to the subject or user the hypoglycemic states of the subject during a reporting period. As shown in FIG. 6, the screen 900 may display annunciations or messages in the reporting period of Sep. 1, 2010 to Sep. 14, 2010. Screen 900 provides annunciation 902 in which an indication of instances of hypoglycemia occurring during overnight fasting is provided to the subject or user as determined exemplarily from the pattern of FIG. 3. Annunciation 904 may also indicate to the subject or user of incidences of hypoglycemia after insulin dosings in the form of bolus doses from the pattern analysis of FIG. 4. Annunciation 906 is also provided to indicate hyperglycemia after exercises as determined from the patterns of FIG. 5. As used herein, the term “user” is intended to indicate primarily a mammalian subject (e.g., a person) who has diabetes but which term may also include a caretaker or a healthcare provider who is operating the meter 10 on behalf of the diabetes subject. As used here, the term “annunciated” and variations on the root term indicate that an announcement may be provided via text, audio, visual or a combination of all modes of communication to a user, a caretaker of the user or a healthcare provider.

It is noted that the various methods described herein can be used to generate software codes using off-the-shelf software development tools such as, for example, Visual Studio 6.0, Windows 2000 Server, and SQL Server 2000. The methods, however, may be transformed into other software languages depending on the requirements and the availability of new software languages for coding the methods. Additionally, the various methods described, once transformed into suitable software codes, may be embodied in any computer-readable storage medium that, when executed by a suitable microprocessor or computer, are operable to carry out the steps described in these methods along with any other necessary steps.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A system for management of diabetes of a subject, the system comprising:
at least one glucose monitor for measurements of the glucose levels of the subject;
an insulin infusion pump configured for communication with the at least one blood glucose monitor and delivery of insulin to the subject; and
a controller in communication with at least the insulin infusion pump and the at least one glucose monitor, the controller being configured to receive or transmit data regarding glucose levels and dosing of insulin from the at least one glucose monitor and pump for analysis the controller so that at least one of a hypoglycemic or hyperglycemic state of the subject is determined by:
evaluation of the glucose measurements measured over distinct fasting periods for hypoglycemia or hyperglycemia during fasting, and in the event a first proportion of such blood glucose measurements measured over distinct fasting periods is less than a hypoglycemic threshold, provide an indication of hypoglycemia over a duration spanning the distinct fasting periods, or alternatively, in the event a second proportion of such blood glucose measurements measured over distinct fasting periods is greater than a hyperglycemic threshold, annunciating an indication of hyperglycemia over a duration spanning the distinct fasting periods; and
ascertainment from the glucose measurements measured after external events have occurred to the user for hypoglycemia or hyperglycemia after such external events and provide an indication of hypoglycemia or hyperglycemia whenever a portion of the blood glucose measurements measured after the external disturbance is at or below a hypoglycemic threshold or whenever the blood glucose measurements measured after the external disturbance is at or above a hyperglycemic threshold.

2. A method for managing diabetes of a subject with at least a blood glucose monitor, the method comprising:
conducting, with the blood glucose monitor, a plurality of blood glucose measurements of the subject;
storing the plurality of blood glucose measurements in a memory;
evaluating the plurality of blood glucose measurements measured over distinct fasting periods for hypoglycemia or hyperglycemia during fasting, and in the event a first proportion of such blood glucose measurements measured over distinct fasting periods is less than a hypoglycemic threshold, annunciating an indication of hypoglycemia over a duration spanning the distinct fasting periods, or alternatively, in the event a second proportion of such blood glucose measurements measured over distinct fasting periods is greater than a hyperglycemic threshold, annunciating an indication of hyperglycemia over a duration spanning the distinct fasting periods.

3. A method for managing diabetes of a subject with at least a blood glucose monitor, the method comprising:
conducting, with at least the blood glucose monitor, a plurality of blood glucose measurements of the subject;
storing the plurality of blood glucose measurements in a memory;
ascertaining from the plurality of blood glucose measurements measured after external events have occurred to the user for hypoglycemia or hyperglycemia after such external events and annunciating an indication of hypoglycemia or hyperglycemia whenever a portion of the blood glucose measurements measured after the external disturbance is at or below a hypoglycemic threshold or whenever the blood glucose measurements measured after the external disturbance is at or above a hyperglycemic threshold.

4. The method of claim 2, further comprising: ascertaining from the plurality of blood glucose measurements measured within a predetermined time after an external event to the user for hypoglycemia or hyperglycemia after such external event and annunciating an indication of hypoglycemia or hyperglycemia whenever a portion of the blood glucose measurements measured after the external event is at or below a hypoglycemic threshold or whenever the blood glucose measurements measured after the external event is at or above a hyperglycemic threshold.

5. The method of claim 2, in which the evaluating comprises the steps of:
determining a number (N1) of blood glucose measurements from the blood glucose measured over distinct fasting periods in which each of the (N1) number of blood glucose measurements is at or above a hypoglycemic threshold;
calculating hypoglycemic value (V1) which is approximately the number (N1) divided by the number of the blood glucose measured over distinct fasting periods and multiplied by 100; and
annunciating hypoglycemia of the user during duration covering the distinct fasting periods whenever the number (N1) is equal to or greater than a first predetermined value and the hypoglycemic value (V1) is equal to or greater than a second predetermined value.

6. The method of claim 2, in which the evaluating comprises the steps of:
determining a number (N2) of blood glucose measurements from the blood glucose measured over distinct fasting periods in which each of the number (N2) of blood glucose measurements is at or above a hyperglycemic threshold;
calculating hyperglycemic value (V2) which is approximately the number (N2) divided by the number of the blood glucose measured over distinct fasting periods and multiplied by 100; and
annunciating hyperglycemia of the user during duration covering the distinct fasting periods whenever the number (N2) is equal to or greater than a third predetermined value and the hyperglycemic value (V2) is equal to or greater than a fourth predetermined value.

7. The method of claim 5, in which the hypoglycemic threshold comprises about 70 mg/dL, the first predetermined value comprises about 2 and the second predetermined value comprises about 5.

8. The method of claim 6, in which the hyperglycemic threshold comprises about 180 mg/dL, the third predetermined value comprises about 3 and the fourth predetermined value comprises about 50.

9. The method of one of claim 3 or claim 4, in which in which the external events comprise an event selected from a group consisting essentially of exercise, psychological stress, physical stress, drug intake, illness, or combinations thereof.

10. The method of claim 9, in which the ascertaining comprises the steps of:
determining from the blood glucose measurements taken within a predetermined time period after the external event in the form of insulin delivery, a first post-insulin BG number (I1) of blood glucose measurements that are at or below a hypoglycemic threshold;
calculating a post-insulin hypoglycemic value (IN1) which is approximately the first post-insulin number (I1) of blood glucose measurements divided by a total number of blood glucose measurements measured after each insulin dosing and multiplied by 100; and
annunciating hypoglycemia of the user after the external events in the form of insulin dosing whenever the hypoglycemic value (IN1) is greater than a predetermined value.

11. The method of claim 9, in which the ascertaining comprises the steps of:
determining from the blood glucose measurements taken within a predetermined time period after insulin dosing, a second post-insulin number (I2) of blood glucose measurements that are at or above a hyperglycemic threshold;
calculating a hyperglycemic value (IN2) which is approximately the second post-insulin number (I2) of blood glucose measurements divided by a total number of blood glucose measurements measured after the insulin dosing and multiplied by 100; and
annunciating hyperglycemia of the user after the external events in the form of insulin dosing whenever the hyperglycemic value (IN2) is greater than another predetermined value.

12. The method of claim 10, in which the hypoglycemic threshold comprises about 70 mg/dL, and the predetermined value comprises about 5.

13. The method of claim 11, in which the hyperglycemic threshold comprises about 180 mg/dL and the another predetermined value comprises about 50.

14. The method of one of claims 10 and 11, in which the predetermined time period after insulin dosing comprises any time period from about 1.5 hours to about 4 hours.

15. The method of claim 9, in which the ascertaining comprises the steps of:
determining from the blood glucose measurements taken within a predetermined time period after each distinct external disturbance a first number (IEE1) of blood glucose measurements that are at or below a hypoglycemic threshold;
calculating a hypoglycemic value (INE1) which is approximately the first number (IEE1) of blood glucose measurements divided by a total number of blood glucose measurements measured after each external disturbance and multiplied by 100; and
annunciating hypoglycemia of the user after external events whenever the hypoglycemic value (INE1) is greater than a predetermined value.

16. The method of claim 9, in which the ascertaining comprises the steps of:
determining from the blood glucose measurements taken within a predetermined time period after each distinct external disturbance a second number (IEE2) of blood glucose measurements that are at or above a hyperglycemic threshold;
calculating a hyperglycemic value (INE2) which is approximately the second number (IEE2) of blood glucose measurements divided by a total number of blood glucose measurements measured after each external disturbance and multiplied by 100; and
annunciating hyperglycemia of the user after external events whenever the hyperglycemic value (INE2) is greater than another predetermined value.

17. The method of claim 15, in which the hypoglycemic threshold comprises about 70 mg/dL, and the predetermined value comprises about 5.

18. The method of claim 16, in which the hyperglycemic threshold comprises about 180 mg/dL and the another predetermined value comprises about 50.

19. The method of one of claim 15 or 16, in which the predetermined time period after each distinct external disturbance comprises about 4 hours.

20. The method of claim 6, in which the blood glucose measurements measured over distinct fasting periods comprise blood glucose measurements tagged by the user as taken during fasting.
